(19) United States
(12) Patent Application Publication
(10) Pub. No.: US 2025/0256108 A1
(43) Pub. Date: Aug. 14, 2025

(54) MANAGEMENT OF STIMULATION AND SENSING ELECTRODE CONFIGURATIONS

(71) Applicant: Boston Scientific Neuromodulation Corporation, Valencia, CA (US)

(72) Inventors: Soroush Niketeghad, Stevenson Ranch, CA (US); G. Karl Steinke, Valencia, CA (US)

(21) Appl. No.: 19/039,347

(22) Filed: Jan. 28, 2025

Related U.S. Application Data
(60) Provisional application No. 63/552,578, filed on Feb. 12, 2024.

(51) Int. Cl. A61N 1/37

(52) U.S. Cl. CPC A61N 1/371

(57) ABSTRACT

A system may include a programmer for a neurostimulator. The neurostimulator may have a plurality of electrodes, and the programmer may include a processor and a memory including instructions which when executed by the processor perform a method that includes: determining a relative geometry between a stimulation electrode configuration and a sensing electrode configuration to reduce a stimulation artifact; using the determined relative geometry to determine the stimulation electrode configuration and the sensing electrode configuration, wherein the stimulation electrode configuration is determined to both stimulate a neural target and cause an evoked potential and the sensing electrode configuration is determined to sense the evoked potential; and programming the neurostimulator with the stimulation electrode configuration and the sensing electrode configuration that have the relative geometry to reduce the stimulation artifact.

### CLAIM OF PRIORITY

This application claims the benefit of U.S. Provisional Application No. 63/552,578, filed on Feb. 12, 2024, which is hereby incorporated by reference in its entirety.

### TECHNICAL FIELD

This document relates generally to medical systems, and more particularly, but not by way of limitation, to systems, devices, and methods for managing evoking and sensing electrode configurations for medical devices that deliver stimulation using a stimulation electrode configuration and that sense evoked responses using a sensing electrode configuration.

### BACKGROUND

Medical devices may include devices configured to deliver a therapy to a patient and/or systems configured to monitor a patient condition via user input and/or sensor(s). For example, these devices may include wearable devices and implantable devices. Some implantable devices may use one or more leads to sense electrical signals or to treat various biological disorders, such spinal cord stimulators (SCS) to treat chronic pain, cortical and Deep Brain Stimulators (DBS) to treat motor and psychological disorders, Peripheral Nerve Stimulation (PNS) including Vagus Nerve Stimulation (VNS), Functional Electrical Stimulation (FES), and other neural stimulators to treat urinary incontinence, sleep apnea, shoulder subluxation, and the like.

It is intended that the stimulation delivered by some medical devices will cause evoked potentials. Some medical devices may attempt to record evoked potentials immediately after delivering stimulation. For example, a DBS therapy may stimulate brain tissue and attempt to record the evoked potentials caused by the stimulation immediately after stimulation. However, the sensed signal may include a stimulation artifact, which may make it difficult to detect the evoked response. Sensing and detecting an evoked potential using a bio-amplifier in an implanted device is particularly challenging when there is a large stimulation artifact because the bio-amplifier may have a limited range and resolution.

### SUMMARY

Various embodiments provide systems and methods to select stimulation and/or sensing configurations to increase or maximize the sensed evoked signal while reducing or minimizing the effect of the stimulation artifact.

An example (e.g., “Example 1”) of a system may include a programmer for a neurostimulator. The neurostimulator may have a plurality of electrodes, and the programmer may include a processor and a memory including instructions which when executed by the processor perform a method that includes: determining at least one stimulation-sensing combination that includes a stimulation electrode configuration for at least some of the plurality of electrodes for use in delivering stimulation that causes an evoked potential and a sensing electrode configuration for at least some of the plurality of electrodes for receiving a sensed signal, wherein the sensed signal includes the evoked potential; evaluating the at least one stimulation-sensing combination for reducing unwanted signal contributions in the sensed signal to provide evaluation results; and programming the neurostimulator with the stimulation electrode configuration and the sensing electrode configuration based on the evaluation results. The evaluation of stimulation-sensing combination(s) may include determining a relative physical, electrical, or combined geometry between a stimulation electrode configuration and a sensing electrode configuration to improve a signal-to-noise ratio by increasing desired signal and/or decreasing unwanted signal(s), such as a stimulation artifact, in the signal. Programming the neurostimulator based on the evaluation results may include various stimulation and/or sensing setting along with the stimulation electrode configuration and the sensing electrode configuration.

In Example 2, the subject matter of Example 1 may optionally be configured such that each of the at least one stimulation-sensing combination is evaluated based on at least one of a relative physical geometry or a relative electrical geometry between the corresponding stimulation electrode configuration and the corresponding sensing electrode configuration.

In Example 3, the subject matter of any of Examples 1-2 may optionally be configured such that the evaluation results include a recommended stimulation-sensing combination with a recommended sensing electrode configuration for a recommended stimulation electrode configuration, wherein the method performed by the processor further includes optimizing at least one of the recommended sensing electrode configuration or the recommended stimulation electrode configuration.

In Example 4, the subject matter of any of Examples 1-3 may optionally be configured such that the stimulation electrode configuration may be configured to provide an electric field with an electric field direction and the sensing electrode configuration is configured to sense the evoked potential to favor a sense direction that is approximately perpendicular to the electric field direction.

In Example 5, the subject matter of Example 4 may optionally be configured such that the neurostimulator includes a lead with the plurality of electrodes organized with longitudinal electrode positions, each longitudinal electrode position includes at least one electrode, and at least one of the longitudinal electrode levels includes segmented electrodes peripherally positioned around the lead in a same longitudinal electrode level. The electric field may be created using electrodes in at least two of the longitudinal electrode positions to generally align the electric field direction along a longitudinal direction of the lead and the determined sensing electrode configuration is configured to use at least two of the segmented electrodes in the same longitudinal electrode level to sense the evoked potential in the sense direction approximately perpendicular to the electric field direction, or the electric field may be created using at least two of the segmented electrodes in the same longitudinal electrode level to generally align the electric field perpendicular to a longitudinal direction of the lead and the determined sensing electrode configuration is configured to use electrodes in at least two of the longitudinal electrode positions to sense the evoked potential in the sense direction approximately along the longitudinal direction of the lead, or the electric field may be created using electrodes in at least two longitudinal electrode positions and at least two segmented electrodes in an electrode level, and the determined sensing electrode configuration is configured to use other electrodes in at least two longitudinal electrode positions and use at least two other segmented electrodes in another electrode level.

In Example 6, the subject matter of Example 4 may optionally be configured such that the neurostimulator includes at least two leads, each of the at least two leads includes a plurality of electrodes organized with longitudinal electrode positions, each longitudinal electrode position includes at least one electrode, and at least one of the plurality of electrodes in each lead is used to either create the electric field or to sense the evoked potential. The electric field may be created using electrodes in one or more of the at least two leads to provide the electric field in the electric field direction to stimulate the neural target. The determined sensing electrode configuration may be configured to use at least two of the electrodes in the at least two leads to sense the evoked potential in the sense direction approximately perpendicular to the electric field direction.

In Example 7, the subject matter of Example 4 may optionally be configured such that the neurostimulator includes a paddle lead that has a plurality of electrodes. At least two of the plurality of electrodes on the paddle lead may be configured to create the electric field in the electric field direction and at least two of the plurality of electrodes on the paddle lead are configured to sense the evoked potential in the sense direction approximately perpendicular to the electric field direction.

In Example 8, the subject matter of any one or more of Examples 1-7 may optionally be configured such that the instructions which when executed by the processor implements a biological model that models evoked potentials for various sensing directions that are evoked when a neural target is stimulated in various electric field directions, and the biological model is implemented to determine the least one the stimulation electrode configuration or the sensing electrode configuration.

In Example 9, the subject matter of any one or more of Examples 1-8 may optionally be configured such that the determined at least one stimulation-sensing combination form acceptable combinations of stimulation and sensing electrode configurations. The method performed by the processor executing the instructions further may include making a list of acceptable stimulation-sensing combinations by determining at least one additional sensing electrode configuration for the stimulation electrode configuration, each of the at least one additional sensing electrode being configured to sense in a corresponding sense direction; and filtering, scoring or ranking the acceptable combinations in the list based on effectively sensing a sensed evoked potential signal and desirably reducing unwanted signal contributions in the sensed signal

In Example 10, the subject matter of Example 9 may optionally be configured such that the list of acceptable combinations is made by determining at least one additional stimulation electrode configuration to create at least one corresponding electric field in at least one corresponding electric field direction for stimulating a neural target, and determining at least one sensing electrode configuration corresponding to each of the at least one additional stimulation configuration to sense in a corresponding at least one sense direction approximately perpendicular to the direction of the corresponding at least one electric field direction for stimulating the neural target.

In Example 11, the subject matter of Example 9 may optionally be configured such that the acceptable stimulation-sensing combinations are ranked by using a biological model to rank the acceptable stimulation-sensing combinations, wherein the biological model is configured to model evoked potentials for various sensing directions that are evoked when a neural target is stimulated in various electric field directions, and wherein the ranking includes using the biological evoked potential model to determine at least one of the stimulation electrode configuration or the sensing electrode configuration to desirably increase a magnitude of the sensed evoked potential signal and desirably reduce unwanted signal contributions in the sensed signal.

In Example 12, the subject matter of Example 9 may optionally be configured such that the neurostimulator includes a lead with the plurality of electrodes organized with longitudinal electrode levels, each longitudinal electrode level includes at least one electrode, and at least one of the longitudinal electrode levels includes segmented electrodes peripherally positioned around the lead in a same longitudinal electrode level, and the list acceptable stimulation-sensing combinations is made by determining different sensing configurations using different combinations of at least two of the segmented electrodes in the same longitudinal electrode level.

In Example 13, the subject matter of Example 12 may optionally be configured such at least a first longitudinal electrode level and a second longitudinal electrode level includes segmented electrodes, and the list of acceptable stimulation-sensing combinations is made by determining different sensing configurations using different combinations of at least two of the segmented electrodes in the first longitudinal electrode level and using different combinations of at least two of the segmented electrodes in the second longitudinal electrode level.

In Example 14, the subject matter of any of Examples 9-13 may optionally be configured such that wherein the method performed by the processor executing the instructions further includes programming a highest ranked acceptable stimulation-sensing combination in the list into the neurostimulator as a current configuration, stimulating the neural target and sensing the evoked potential using the programmed neurostimulator, and determining whether the sensed evoked potential is within an input range of a bio-amplifier used to amplify the sensed evoked potential.

In Example 15, the subject matter of Example 14 may optionally be configured such that the sensed evoked potential is determined to not be within the input range of the bio-amplifier. The method performed by the processor executing the instructions may further include removing the current configuration from the list and programming a remaining highest ranked acceptable stimulation-sensing combination in the list into the neurostimulator as a subsequent current configuration.

Example 16 includes subject matter (such as a method, means for performing acts, machine readable medium including instructions that when performed by a machine cause the machine to perform acts, or an apparatus to perform). The subject matter may include determining at least one stimulation-sensing combination that includes a stimulation electrode configuration for use in delivering stimulation that causes an evoked potential and a sensing electrode configuration for receiving a sensed signal, wherein the sensed signal includes the evoked potential; evaluating the at least one stimulation-sensing combination for reducing unwanted signal contributions in the sensed signal to provide evaluation results; and programming a neurostimulator with the stimulation electrode configuration and the sensing electrode configuration based on the evaluation results.

In Example 17, the subject matter of Example 16 may optionally be configured such that each of the at least one stimulation-sensing combination is evaluated based on at least one of a relative physical geometry or a relative electrical geometry between the corresponding stimulation electrode configuration and the corresponding sensing electrode configuration.

In Example 18, the subject matter of any of Examples 16-17 may optionally be configured such that the evaluation results include a recommended stimulation-sensing combination with a recommended sensing electrode configuration for a recommended stimulation electrode configuration, wherein the method further includes optimizing at least one of the recommended sensing electrode configuration or the recommended stimulation electrode configuration.

In Example 19, the subject matter of any of Examples 16-18 may optionally be configured to further include stimulating the neural target using the neurostimulator programmed with the determined stimulation electrode configuration and sensing the evoked potential using the neurostimulator programmed with the determined sensing electrode configuration.

In Example 20, the subject matter of any of Examples 16-19 may optionally be configured such that the neurostimulator is programmed with the determined stimulation electrode configuration and the determined sensing electrode configuration where the programmed neurostimulator is configured to stimulate the neural target using the determined stimulation electrode configuration and sense the evoked potential using the determined sensing electrode configuration. The neurostimulator may include a lead with a plurality of electrodes organized with longitudinal electrode levels, each longitudinal electrode level includes at least one electrode, and at least one of the longitudinal electrode levels includes segmented electrodes peripherally positioned around the lead in a same longitudinal electrode level. An electric field may be created using electrodes in at least two of the longitudinal electrode positions to generally align an electric field direction along a longitudinal direction of the lead. The sensing electrode configuration may be configured to use at least two of the segmented electrodes in the same longitudinal electrode level to sense the evoked potential in the sense direction approximately perpendicular to the electric field direction.

In Example 21, the subject matter of any of Examples 16-20 may optionally be configured such that the neurostimulator includes at least two leads, each of the at least two leads including a plurality of electrodes organized with longitudinal electrode positions, and each longitudinal electrode position includes at least one electrode. At least one of the plurality of electrodes in each lead may be used to either create an electric field or to sense the evoked potential. The electric field may be created using electrodes in one or more of the at least two leads to provide the electric field in the electric field direction to stimulate the neural target. The determined sensing electrode configuration is configured to use at least two of the electrodes in the at least two leads to sense the evoked potential in the sense direction approximately perpendicular to the electric field direction.

In Example 22, the subject matter of any one or more of Examples 16-17 may optionally be configured such that the neurostimulator includes a paddle lead, the paddle lead including a plurality of electrodes, and at least two of the plurality of electrodes on the paddle lead are configured to create an electric field in an electric field direction and at least two of the plurality of electrodes on the paddle lead are configured to sense the evoked potential in the sense direction approximately perpendicular to the electric field direction.

In Example 23, the subject matter of any one or more of Examples 16-22 may optionally be configured such that the least one the stimulation electrode configuration or the sensing electrode configuration is determined using a biological model. The biological model may be configured to model evoked potentials for various sensing directions that are evoked when a neural target is stimulated in various electric field directions.

In Example 24, the subject matter of any one or more of Examples 16-23 may optionally be configured such that the determined at least one stimulation-sensing combination form acceptable combinations of stimulation and sensing electrode configurations. The method may include making a list of acceptable stimulation-sensing combinations by determining at least one additional sensing electrode configuration for the stimulation electrode configuration, each of the at least one additional sensing electrode being configured to sense in a corresponding sense direction, and filtering, scoring or ranking the acceptable combinations in the list based on effectively sensing a sensed evoked potential signal and desirably reducing unwanted signal contributions in the sensed signal.

In Example 25, the subject matter of Example 24 may optionally be configured such that the making the list of acceptable stimulation-sensing combinations includes determining at least one additional stimulation electrode configuration to create at least one corresponding electric field in at least one corresponding electric field direction for stimulating a neural target, and determining at least one sensing electrode configuration corresponding to each of the at least one additional stimulation configuration to sense in a corresponding at least one sense direction approximately perpendicular to the direction of the corresponding at least one electric field direction for stimulating the neural target.

In Example 26, the subject matter of any one or more of Examples 24-25 may optionally be configured such that the ranking the acceptable stimulation-sensing combinations includes using a biological model to rank the acceptable stimulation-sensing combinations. The biological model may be configured to model evoked potentials for various sensing directions that are evoked when a neural target is stimulated in various electric field directions. The ranking may include using the biological model to determine at least one of the stimulation electrode configurations or the sensing electrode configuration to desirably increase a magnitude of the sensed evoked potential signal and desirably reduce unwanted signal contributions in the sensed signal.

In Example 27, the subject matter of any one or more of Examples 24-26 may optionally be configured such that the neurostimulator includes a lead with a plurality of electrodes organized with longitudinal electrode levels, each longitudinal electrode level includes at least one electrode, and at least one of the longitudinal electrode levels includes segmented electrodes peripherally positioned around the lead in a same longitudinal electrode level; and the making the list of acceptable stimulation-sensing combinations includes determining different sensing configurations using different combinations of at least two of the segmented electrodes in the same longitudinal electrode level.

In Example 28, the subject matter of Example 27 may optionally be configured such that at least a first longitudinal electrode level and a second longitudinal electrode level includes segmented electrodes, and the making the list of acceptable stimulation-sensing combinations includes determining different sensing configurations using different combinations of at least two of the segmented electrodes in the first longitudinal electrode level and using different combinations of at least two of the segmented electrodes in the second longitudinal electrode level.

In Example 29, the subject matter of any one or more of Examples 24-26 may optionally be configured to further include programming a highest ranked acceptable stimulation-sensing combination in the list into the neurostimulator as a current configuration, stimulating the neural target and sensing the evoked potential using the programmed neurostimulator, and determining whether the sensed evoked potential is within an input range of a bio-amplifier used to amplify the sensed evoked potential.

In Example 30, the subject matter of Example 29 may optionally be configured such that the sensed evoked potential is determined to not be within the input range of the bio-amplifier. the method may further include removing the current configuration from the list and programming a remaining highest ranked acceptable stimulation-sensing combination in the list into the neurostimulator as a subsequent current configuration.

In Example 31, the subject matter of any one or more of Examples 16-30 may optionally be configured such that the programmed stimulation electrode configuration is configured to provide an electric field with an electric field direction and the programmed sensing electrode configuration is configured to sense the evoked potential to favor a sense direction that is approximately perpendicular to the electric field direction.

Example 32 includes subject matter that includes non-transitory machine-readable medium including instructions, which when executed by a machine, cause the machine to perform a method. The method performed by the machine may include determining at least one stimulation-sensing combination that includes a stimulation electrode configuration for use in delivering stimulation that causes an evoked potential and a sensing electrode configuration for receiving a sensed signal, wherein the sensed signal includes the evoked potential; evaluating the at least one stimulation-sensing combination for reducing unwanted signal contributions in the sensed signal to provide evaluation results; and programming a neurostimulator with the stimulation electrode configuration and the sensing electrode configuration based on the evaluation results.

In Example 33, the subject matter of Example 32 may optionally be configured such that the method further includes the neurostimulator includes at least two leads, each of the at least two leads including a plurality of electrodes organized with longitudinal electrode positions, each longitudinal electrode position includes at least one electrode, and at least one of the plurality of electrodes in each lead is used to either create the electric field or to sense the evoked potential.

In Example 34, the subject matter of any one or more of Examples 32-33 may optionally be configured such that the least one the stimulation electrode configuration or the sensing electrode configuration is determined using a biological model. The biological model may be configured to model evoked potentials for various sensing directions that are evoked when the neural target is stimulated in various electric field directions.

In Example 35, the subject matter of any one or more of Examples 32-34 may optionally be configured such that determined at least one stimulation-sensing combination form acceptable combination of stimulation and sensing electrode configuration. The method may further include making a list of acceptable stimulation-sensing combinations by determining at least one additional sensing electrode configuration for the stimulation electrode configuration, each of the at least one additional sensing electrode being configured to sense in a corresponding sense direction; and filtering, scoring or ranking the acceptable combinations in the list based on effectively sensing a sensed evoked potential signal and desirably reducing unwanted signal contributions in the sensed signal.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrical stimulation system.

FIG. 5 illustrates, by way of example and not limitation, a more generalized example of a medical system that includes a medical device and a processing system.

FIG. 6 illustrates, by way of example, an example of an electrical therapy-delivery system.

FIG. 7 illustrates, by way of example and not limitation, a monitoring system and/or the electrical therapy-delivery system of FIG. 6, implemented using an IMD.

FIG. 8 illustrates, by way of example and not limitation, a problem with using bio-amplifiers with a limited input range that is saturated by stimulation artifact to sense an evoked response.

FIGS. 9A-9B illustrate, by way of example and not limitation, available electrodes that are available for selection to deliver the neurostimulation and are available for selection to sense a biological evoked response.

FIG. 10 illustrates, by way of example and not limitation, a neurostimulation program that includes a parameter set defining a stimulation electrode configuration for delivering neurostimulation and defining a sensing electrode configuration for sensing an evoked potential.

FIG. 11 illustrates, by way of example and not limitation, a sensing direction for a sensing configuration and electrical field direction for an electrical field.

FIG. 12 illustrates an example of an electric field generated using electrodes on a lead and a biological electric field in which an evoked response may be sensed.

FIG. 13 illustrates a relationship between detectable evoked potential amplitudes and stimulation artifact amplitudes bounded by a maximum input for a bio-amplifier for the three sensing configuration examples illustrated in FIG. 12.

FIG. 14 illustrates, by way of example and not limitation, a method for determining the sensing electrode configuration and the stimulation electrode configuration.

FIG. 15 illustrates, by way of example and not limitation, a method for stimulating a neural target using a determined relative geometry between a stimulation electrode configuration and a sensing electrode configuration to reduce a stimulation artifact.

FIG. 16 illustrates, by way of example and not limitation, a method for filtering, scoring or ranking acceptable combinations of stimulation electrode configurations and sensing electrode configurations.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized, and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

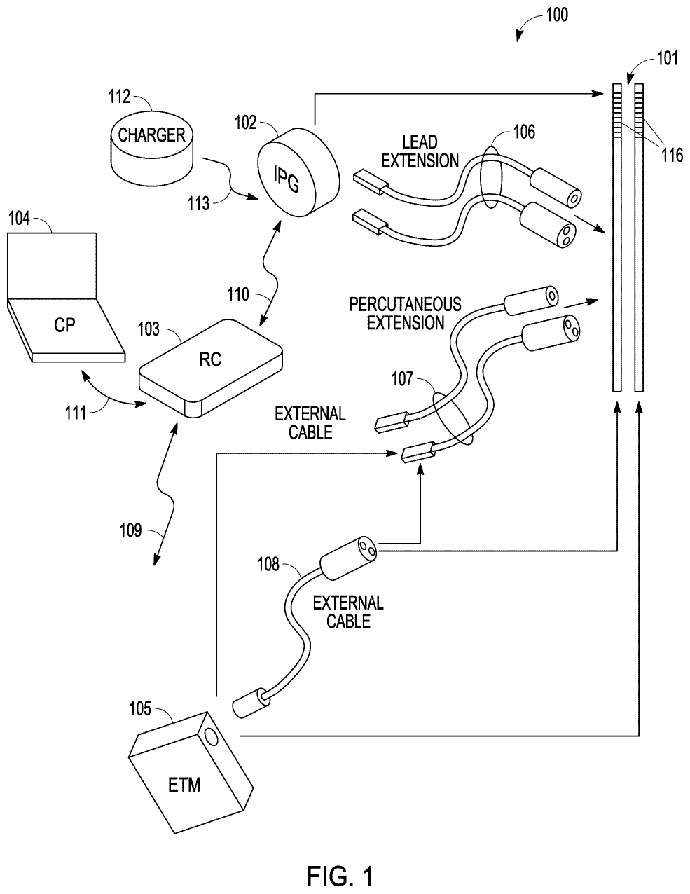

FIG. 1 illustrates, by way of example and not limitation, an electrical stimulation system 100, such as may be used to deliver DBS. The electrical stimulation system 100 may generally include a one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Other types of leads may be used. The IPG 102 includes pulse generation circuitry that delivers electrical modulation energy in the form of a pulsed electrical waveform (i.e., a temporal series of electrical pulses) to the electrodes in accordance with a set of modulation parameters. The leads may be directional leads with a lead marker for use to help determine an orientation for an implanted lead. Directional leads may be used to target neural stimulation for DBS, SCS, PNS or other electrical stimulation.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. The ETM 105 may be used for situations where a brief period of therapy is suitable to achieve the desired effects. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned ON or OFF and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneously charge the IPG 102 via a wireless link such as an inductive link 113. Once the IPG 102 has been programmed, the IPG 102 may function as programmed without the RC 103 or CP 104 being present. It is noted that some IPGs do not require charging, as some are manufactured with primary batteries with sufficient capacity to provide therapy over a clinically useful duration without recharging.

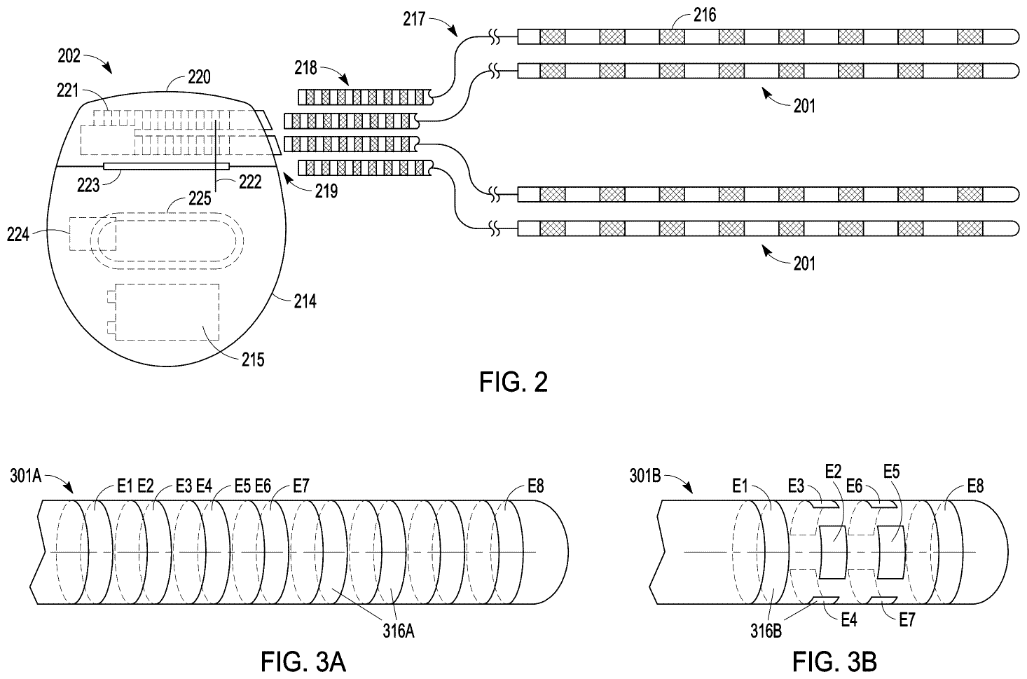

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 can also lack a battery and can be wirelessly powered by an external source. The IPG 202 may be coupled to one or more leads, such as leads 201 as illustrated herein. The leads 201 can each include a plurality of electrodes 216 for delivering electrostimulation energy, recording electrical signals, or both. In some examples, the leads 201 can be rotatable so that the electrodes 216 can be aligned with the target neurons after the neurons have been located such as based on the recorded signals. The electrodes 216 can include one or more ring electrodes, and/or one or more sets of segmented electrodes (or any other combination of electrodes), examples of which are discussed below with reference to FIGS. 3A and 3B.

The leads 201 can be implanted near or within the desired portion of the body to be stimulated. In an example of operations for DBS, access to the desired position in the brain can be accomplished by drilling a hole in the patient's skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracting the lead.

Lead wires 217 within the leads may be coupled to the electrodes 216 and to proximal contacts 218 insertable into lead connectors 219 fixed in a header 220 on the IPG 202, which header can comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 can include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) can be tunneled through and under the neck and the scalp, with the electrodes 216 implanted through holes drilled in the skull and positioned for example in the subthalamic nucleus (STN) and the pedunculopontine nucleus (PPN) in each brain hemisphere. The IPG 202 can also be implanted underneath the scalp closer to the location of the electrodes' implantation. The leads 201, or the extensions, can be integrated with and permanently connected to the IPG 202 in other solutions.

Stimulation in IPG 202 is typically provided by pulses each of which may include one phase or multiple phases. For example, a monopolar stimulation current can be delivered between a lead-based electrode (e.g., one of the electrodes 216) and a case electrode. A bipolar stimulation current can be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes can either be used (an active electrode) or unused (OFF). When the electrode is used, the electrode can be used as an anode or cathode and carry anodic or cathodic current. In some architectures, electrodes of the same polarity can deliver distinct amounts of current simultaneously using multiple electrical sources, to provide greater control of the electric field. In some instances, an electrode might be an anode for a period of time and a cathode for a period of time (e.g., when multiple phases are used, for example, for charge recovery or other purposes). These and possibly other stimulation parameters taken together comprise a stimulation program that the stimulation circuitry 224 in the IPG 202 can execute to provide therapeutic stimulation to a patient.

In some examples, a measurement device coupled to the muscles or other tissue stimulated by the target neurons, or a unit responsive to the patient or clinician, can be coupled to the IPG 202 or microdrive motor system. The measurement device, user, or clinician can indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 can direct stimulus current to a selected angular range around the lead.

Segmented electrodes can typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering using multiple electrical sources can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be together with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length of the lead

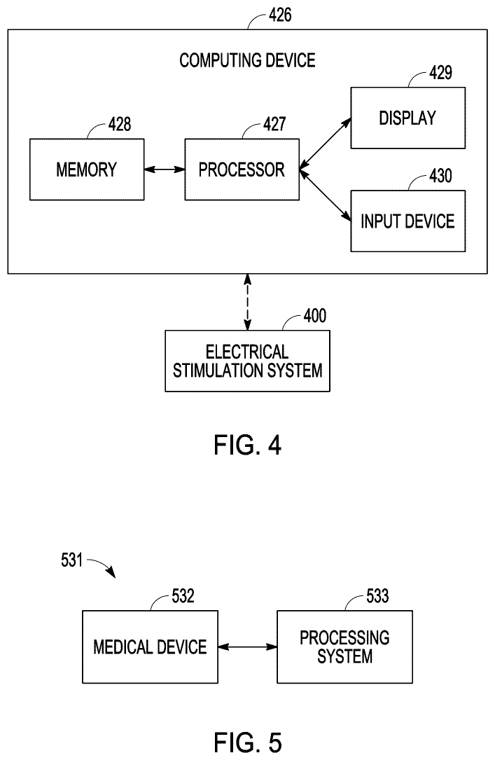

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1. Alternatively, the computing device 426 may be integrated with the electrical stimulation system 100, such as part of the IPG 102, RC 103, CP 104, or ETM 105 illustrated in FIG. 1.

The computing device 426, also referred to as a programming device, can be a computer, tablet, mobile device, or any other suitable device for processing information. The computing device 426 can be local to the user or can include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 can include a watch, wristband, smartphone, or the like. Such computing devices can wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 can be a wearable device used by the patient only during programming sessions. Alternatively, the computing device 426 can be worn all the time and continually or periodically adjust the stimulation parameters. In an example, a closed-loop algorithm for determining or updating stimulation parameters can be implemented in a mobile device, such as a smartphone, which is connected to the IPG or an evaluating device (e.g., a wristband or watch). These devices can also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses. In various examples, portions of the functions of the processor 427 may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein. The memory 428 can store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information, and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and can include a printer. The display 429 may be a part of a user interface configured to display information about stimulation settings (e.g., electrode configurations and stimulation parameter values and value ranges) and user control elements for programming a stimulation setting into an IPG.

The input device 430 may be, for example, a keyboard, mouse, touch screen, track ball, joystick, voice recognition system, or any combination thereof, or the like. Another input device 430 may be a camera from which the clinician can observe the patient. Yet another input device 430 may a microphone where the patient or clinician can provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user can provide information between the electrical stimulation system 400 and the computing device 426 using a computer-readable medium or by some other mechanism.

FIG. 5 illustrates, by way of example and not limitation, a more generalized example of a medical system 531 that includes a medical device 532 and a processing system 533. For example, the electrical stimulation system 400 of FIG. 4 may be a more specific example of the medical device 532 of FIG. 5, and computing device 426 of FIG. 4 may be a more specific example of the processing system 533 of FIG. 5. The medical device may be configured to use at least one directional lead to provide sensing functions and/or therapy functions. For example, the medical device may include a device configured to use a parameter set to deliver an electrical stimulation therapy. The medical device may be an implantable medical device such as an implantable neurostimulator. The implantable medical device may be configured to deliver SCS or DBS therapy. The medical device may include more than one medical device. The processing system may be within a single device or may be a distributed system across two or more devices including local and/or remote systems. According to various embodiments, the medical system may include at least one medical device configured to treat a condition by delivering a therapy to a patient.

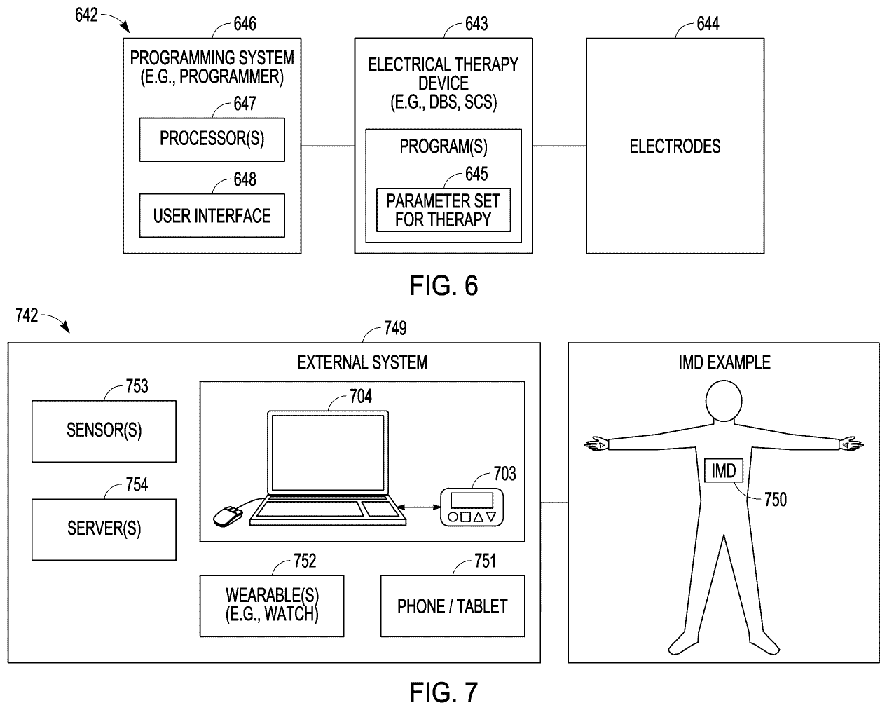

FIG. 6 illustrates, by way of example, an example of an electrical therapy-delivery system. The illustrated system 642 may be a more specific example of the system illustrated in FIG. 5 or form a portion of the system illustrated in FIG. 5. The illustrated system 642 includes an electrical therapy device 643 configured to deliver an electrical therapy to electrodes 644 to treat a condition in accordance with a programmed parameter set 645 for the therapy. The system 642 may include a programming system 646, which may function as at least a portion of a processing system, which may include one or more processors 647 and a user interface 648. The programming system 646 may be used to program and/or evaluate the parameter set(s) used to deliver the therapy. The illustrated system 642 may be a DBS system. In some embodiments, the illustrated system 642 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameters sets through a computerized programming system to allow the optimum modulation parameters to be determined based on patient feedback or other means and to subsequently program the desired modulation parameter sets.

FIG. 7 illustrates, by way of example and not limitation, the electrical therapy-delivery system of FIG. 6 implemented using an IMD. The IMD may include a DBS stimulator. The illustrated system 742 includes an external system 749 that may include at least one programming device. The illustrated external system 749 may include a clinician programmer 704, similar to CP 104 in FIG. 1, configured for use by a clinician to communicate with and program the neuromodulator, and a remote-control device 703, similar to RC 103 in FIG. 1, configured for use by the patient to communicate with and program the neuromodulator. For example, the remote-control device 703 may allow the patient to turn a therapy ON and OFF, change or select programs, adjust patient-programmable parameter(s) of the plurality of modulation parameters, and/or provide inputs used to detect event(s). The patient may use custom external devices to perform specific tasks useful for the event-triggered therapy of that patient. The external devices may communicate directly with the IPG/IMD, or they may communicate with an intermediary device (such as an RC) which in turn communicates with the IPG/IMD. FIG. 7 illustrates an IMD 750, although the monitor and/or therapy device may be an external device such as a wearable device. The external system 749 may include a network of computers, including computer(s) remotely located from the IMD 750 that are capable of communicating via one or more communication networks with the programmer 704 and/or the remote-control device 703. The remotely located computer(s) and the IMD 750 may be configured to communicate with each other via another external device such as the programmer 704 or the remote-control device 703. The remote-control device 703 and/or the programmer 704 may allow a user (e.g., patient, caregiver and/or clinician or rep) to answer questions as part of a data collection process. The external system 749 may include personal devices such as a phone or tablet 751, wearables such as a watch 752, sensor(s) 753 and server(s) s 754. The watch may include sensor(s), such as sensor(s) for detecting activity, motion and/or posture. Other wearable sensor(s) may be configured for use to detect activity, motion and/or posture of the patient. The external system 749 may include, but is not limited to, a phone and/or a tablet. The system 742 may include medical record(s) for the patient and broader patient population(s). The medical record(s) may be stored and accessed using one or more servers (e.g., local or remote servers such as cloud-based servers). The external device may also include device(s) (e.g., app on phone/tablet or a custom device) used by the patient to perform tasks and may also monitor the ability of the patient to perform the task. The external system may be used to process inputs, detect events, analyze the results and/or optimize the training. Processing may be done using cloud computing, fog computing, and/or edge computing. Cloud computing may include a network of devices or servers connected over the Internet. Cloud computing may have very large storage space and processing capabilities. However, cloud computing can have higher latencies. Fog computing occurs physically closer to the end user compared to centralized data centers. The infrastructure of fog computing may connect end devices with central servers in the cloud. Fog computing may provide lower latency for quicker responses and may use other communication technology other than the Internet. Edge computing is done at the device level. The processing for different functions may be distributed over multiple devices and may be distributed over edge computing, fog computing and cloud computing.

Evoked potentials are evoked by stimulation. Thus, evoked potential recordings occur almost immediately after the stimulation of neural tissue. Evoked potentials may be recorded after stimulation of brain tissue, spinal cord tissue or other tissue near the spinal cord, or other neural tissue.

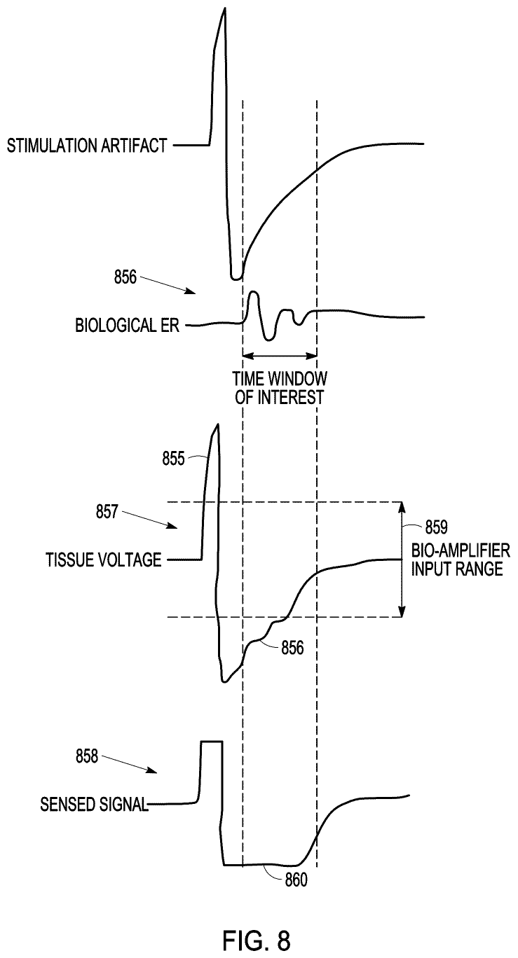

FIG. 8 illustrates, by way of example and not limitation, a problem with using bio-amplifiers with a limited input range that is saturated by stimulation artifact to sense an evoked response. The figure illustrates a stimulation artifact waveform 855 from an electrical stimulation pulse, a biological evoked response signal 856 that is desired to be sensed, a tissue voltage 857 from the combination of the stimulation artifact and the biological evoked response, and a sensed signal 858 sensed by a bio-amplifier with a limited range. The bio-amplifier is attempting to sense the biological evoked response 856 shortly after the stimulation, but the evoked response 856 occurs during a time frame in which the much larger stimulation artifact 855 also occurs and overpowers the smaller biological evoked response 856. If the bio-amplifier has a wide enough range to handle the amplitude of the stimulation artifact, then the sensor may detect the biological evoked response signal 856 superimposed on the stimulation artifact 855, and the biological evoked response signal 856 may be extracted via post processing. In the illustrated figure, the biological evoked response of interest would be found during the negative peak of the sensed signal. However, some bio-amplifiers, such as amplifiers incorporated into implanted devices, may have a limited bio-amplifier input range 859 which may be attributed to a limited power supply voltage or other limits of the amplifier circuit. This limited input range causes the amplifier to clip the positive and negative peaks of the sensed signal. A result may be that the biological evoked response may be greatly distorted and even completely lost (see signal portion 860) when peaks are clipped as shown in the sensed signal 858.

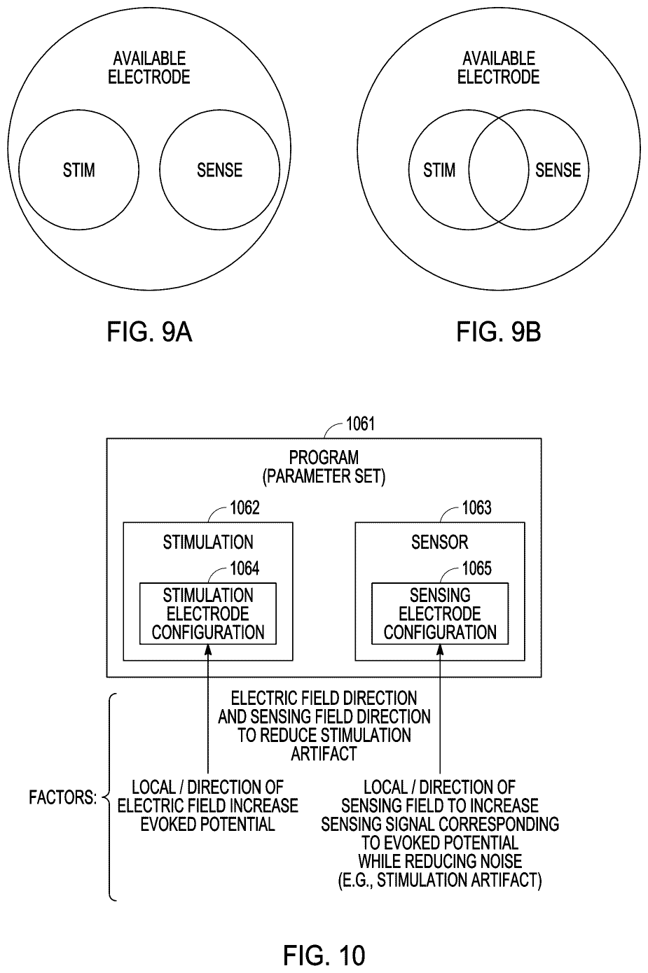

FIGS. 9A-9B illustrate, by way of example and not limitation, available electrodes that are available for selection to deliver the neurostimulation and are available for selection to sense a biological evoked response. Generally, more electrodes provide more options for delivering a stimulation electric field at a desired location and in a desired orientation, and also provide more options for sensing configurations with desired sensing vector(s). The electrodes may be incorporated into various electrode arrangement(s), and may be incorporated on one or more different types of leads. For example, an electrode arrangement may be incorporated on a paddle lead, on one or more percutaneous lead(s) with ring electrode(s) and/or segmented electrodes, and/or using transcutaneous electrodes. For example, the use of more than one lead may provide additional electrode(s) that may be selected for use to create electric field vectors and/or may be selected for use to create sensing vectors. In some embodiments as illustrated in FIG. 9A, the stimulation electrode configuration used to deliver the electrical field may only use electrodes that are not used in the sensing electrode configuration to sense the biological evoked response signal. In some embodiments as illustrated in FIG. 9B, the stimulation electrode configuration used to deliver the electrical field and the sensing electrode configuration used to sense the biological evoked response may use at least some of the same electrodes.

FIG. 10 illustrates, by way of example and not limitation, a neurostimulation program that includes a parameter set defining a stimulation electrode configuration for delivering neurostimulation and defining a sensing electrode configuration for sensing an evoked potential. A neurostimulator may be programmed with one or more programs 1061, and each of the programs has a parameter set used to define the stimulation and sensing performed by the neurostimulator. The parameter set for the program may include a stimulation parameter subset 1062 and a sensing parameter subset 1063. The stimulation parameter subset 1062 includes, among other things, a stimulation electrode configuration 1064. The stimulation electrode configuration may include a selection of electrodes that are active, a selection of the active electrodes that are configured as cathodes and a selection of the active electrodes that are configured as anodes. The stimulation electrode configuration may include fractionalization values controlling the percentage of the anodic energy delivered through individual ones of the anodes and controlling the percentage of the cathodic energy delivered through individual ones of the cathodes. The stimulation parameter subset 1062 may include other stimulation parameters such as waveform shape, amplitude, pulse width, pulse frequency, burst duration for a burst of pulses, burst frequency for bursts of pulses, On times, and Off times. The stimulation parameter subset may include parameters for multiple channels of neurostimulation.

The sensing parameter subset 1063 includes, among other things, a sensing electrode configuration 1065. The sensing electrode configuration 1065 may include the electrode(s) used as a first reference (e.g., positive) and the electrode(s) used as a second reference (e.g., negative). The sensing electrode configuration controls a sensing direction for the sensing parameter subset, and the stimulation electrode configuration controls an electric field direction for the stimulation field.

The present subject matter determines the stimulation electrode configuration, or the sensing electrode configuration, or both the stimulation and sensing electrode configurations to increase the sensed signal while reducing the contribution of the stimulation artifact. Factors used to determine the stimulation electrode configuration 1064 may include the location and/or direction of the electric field to increase an evoked potential and the stimulation electrode configuration. Factors used to determine the sensing electrode configuration 1065 may include the location and/or direction of the sensing signal to increase the evoked potential while reducing noise such as the noise affiliated with the stimulation artifact. The electric field direction and the sensing field direction are generally selected to reduce the stimulation artifact while still being to deliver the neural stimulation to the targeted tissue and sense the evoked response to the stimulation. For example, the sensing electrode configuration may provide a sensing vector generally perpendicular to the stimulation electrical field. Another factor contributing to the determination of the sensing and/or stimulation electrode configuration is a biological electric field. This field may be modeled to represent the tissue that produces the evoked response to the neural stimulation. An example of such evoked response is evoked resonant neural activity (ERNA) within the brain. The sensing vector for the sensing configuration may be chosen to enhance the sensing of the evoked response within the biological electrical field.

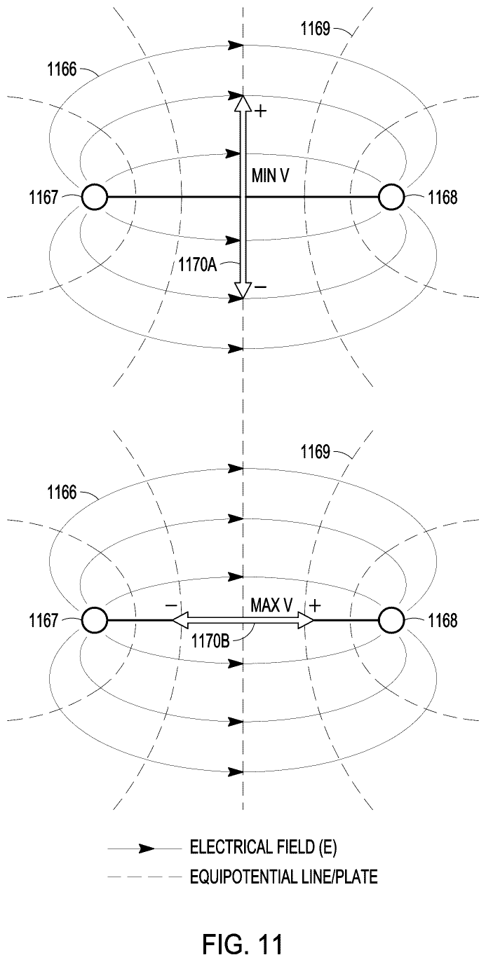

FIG. 11 illustrates, by way of example and not limitation, different options of a sensing direction for a sensing configuration and electrical field direction for an electrical field. A stimulation electric field 1166 may be generated between a positive electrode (anode) 1167 and negative electrode (cathode) 1168. The dotted lines represent equipotential lines 1169 within the stimulation electric field 1166. A sensing vector 1170A may be created using a first reference electrode (e.g., positive) and a second reference electrode (e.g., negative). The illustrated sensing vector 1170A may be perpendicular to the electrical field, which is along an equipotential line of the electric field. As a result, the contribution of the stimulation electric field 1166 to the voltage of the sensed signal is minimal as it is along the equipotential line. However, if the sensing vector 1170B is in line with the stimulation electric field 1166, crossing equipotential lines within the electric field, the contribution of the stimulation electric field 1166 to the voltage of the sensed signal is maximal.

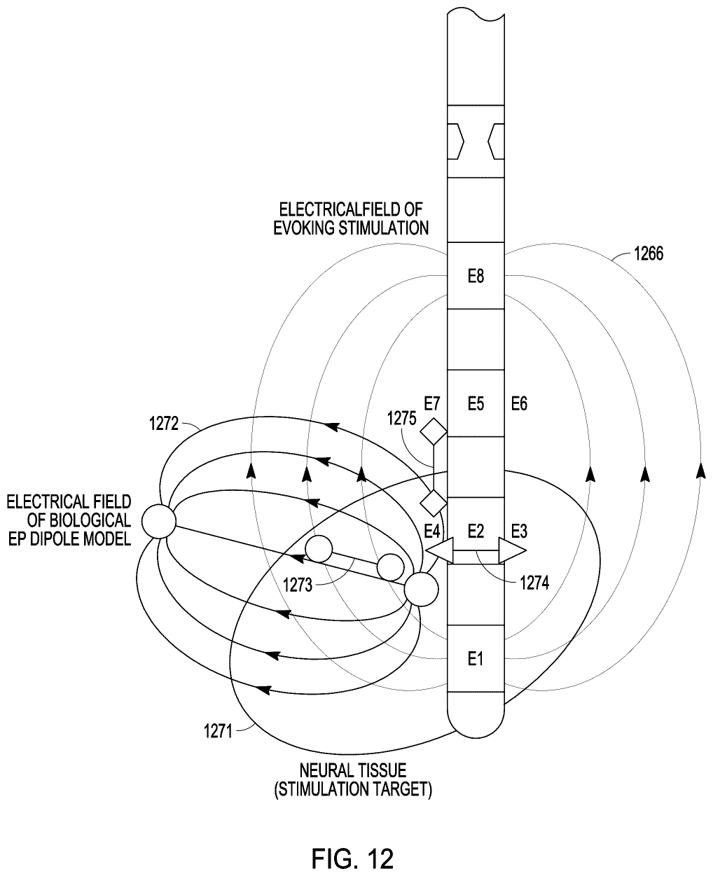

FIG. 12 illustrates an example of an electric field generated using electrodes on a lead and a biological electric field in which an evoked response may be sensed. The lead-generated electric field 1266 may be delivered to targeted neural tissue 1271 and may evoke a response such as a biological electrical field 1272. By way of example and not limitation, evoked resonant neural activity (ERNA) in the brain is an example of an evoked response to DBS. In the illustrated embodiment, the lead-generated electric field 1266 is generated using electrodes E1 and E8. It is understood that other electrode combinations may be used to create an electric field. The lead-generated field produces the stimulation artifact. The biological electrical field 1272 (e.g., ERNA), which may be modeled, is the biological evoked response. It may be to increase sensing of the biological electrical field 1272 while reducing the stimulation artifact in the sensed signal attributed to the lead-generated field 1266. FIG. 12 also illustrates, by way of example and not limitation, three sensing vectors for a sensing configuration. The first sensing electrode configuration, illustrated as a first sensing vector 1273 with circle endpoints, is in line with the biological electric field. The second sensing electrode configuration, e.g., illustrated as a second sensing vector 1274 with arrow endpoints, is perpendicular to the lead-generated electric field. The third sensing electrode configuration, e.g., illustrated as a third sensing vector 1275 with diamond endpoints, is parallel to the lead generated electric field.

It is noted that the model can include various aspects of relevant physics, and may or may not need to produce evoked potentials. Systems may be developed to make a series of measurements to finetune the configuration of the model, which may then be used to do filtering, make predictions or recommendations about the stimulation electrode configuration and/or the sensing electrode configuration.

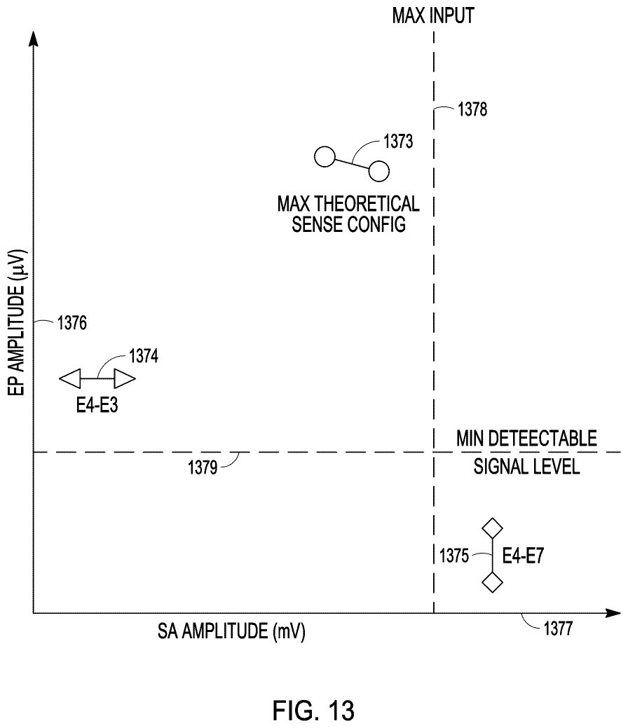

FIG. 13 illustrates a relationship between detectable evoked potential amplitudes 1376 and stimulation artifact amplitudes 1377 bounded by a maximum input for a bio-amplifier for the three sensing configuration examples illustrated in FIG. 12. As noted previously (e.g., FIG. 8), amplifiers incorporated into implantable medical devices may have a limited range, such that there is a maximum input 1378 for the bio-amplifier above which the amplifier may be saturated and the signal may be clipped. This maximum input is illustrated as the maximum stimulation artifact, as the stimulation artifact amplitude (mV) 1377 is orders of magnitude greater than the evoked potential amplitude (μV) 1376. Also illustrated is the minimum detectable signal level (μV) 1379 for the amplitude of the evoked potential.

The goal may be to maximize the sensing of the evoked response in the biological electrical field (FIG. 12, 1273) and minimizing the stimulation artifact from the lead-generated electric field (FIG. 12, 1266). If there were electrodes aligned with the biological electrical field that could be selected for the first sensing electrode configuration (FIG. 12, 1273), the aligned electrodes would be the maximum theoretical sensing configuration for the sensing the biological electrical field. FIG. 13 illustrates that this first sensing electrode configuration 1373 provides a sensed signal that is less than the maximum input 1378 for the bio-amplifier and yet is well above the minimum detectable signal level 1379. If electrodes E4-E3 are selected for the second sensing electrode configuration (FIG. 12, 1274), FIG. 13 illustrates that the second sensing electrode configuration 1374 provides a sensed signal that is below the maximum input 1378 for the bio-amplifier and is above the minimum detectable signal level 1379 for the evoked potential. For example, the sensing vector created using electrodes E2/E3, E3/E4, or E4/E2 may be perpendicular to the direction of the electrical field generated using the E1/E8 electrodes. If electrodes E4-E7 are selected for the third sensing electrode configuration (FIG. 12, 1275), FIG. 13 illustrates that the third sensing electrode configuration 1375 provides a sensed signal that is over the maximum input 1378 for the bio-amplifier and the below the minimum detectable signal level 1379 for the evoked potential.

The model for the biological field may be patient specific based on the imaging that is provided prior to the surgery. For example, MRI images of the patient prior to the surgery are typically available. The physiological model may be based on MRI. After implantation, a CT scan may be performed. The combination of the CT scan the MRI scan may be used to estimate the lead location and orientation, the electric field generated by the lead, and the biological electrical field evoked by the lead-generated electric field. A general template of a physiological model may be mapped to the patient based on the MRI as the MRI may provide an indication of where and which orientation of the biological electrical field model. A goal is to quickly determine an appropriate sensing and stimulation configurations to increase the sensed signal and reduce the stimulation artifact in the signal. However, measurement(s) may be taken to refine the model. Classes of models may be used to try to avoid empirical measurement increasing the speed of finding desired (e.g., optimum) sensing and stimulation configurations.

Some embodiments work just with the stimulating response without the use an electrical model. If there are only a few electrodes like those shown in this figure, there are only a few options that can be chosen for sensing. In such situations, the system may select a sensing configuration that has a sensing vector generally perpendicular to the direction of the stimulation electric field. However, if there are more electrodes available for selection for sensing, then the sensing configuration can be fine tuned to be more specific to the model.

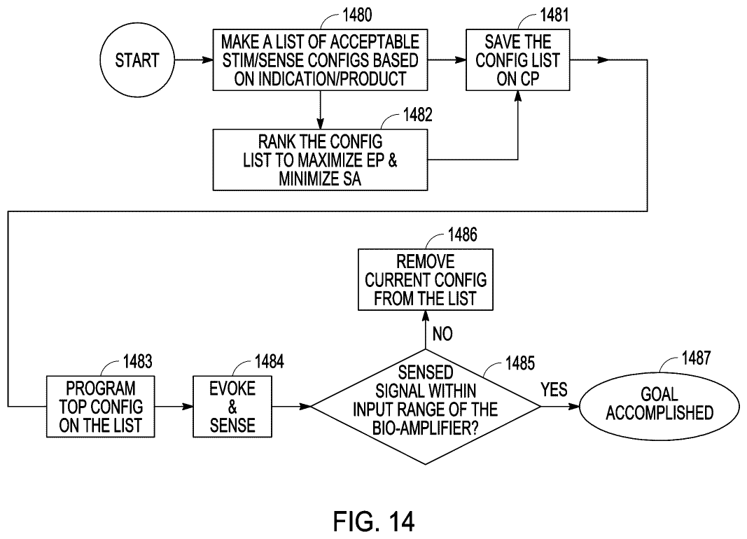

FIG. 14 illustrates, by way of example and not limitation, a method for determining the sensing electrode configuration and the stimulation electrode configuration. A list of acceptable stimulation and sensing configurations may be made based on the indication and product 1480. The configuration list may be saved on a clinician programmer 1481. However, there may be very many configurations to try and there is very little time during surgery to try these configurations. Various embodiments filter, score or rank the configuration list for favoring combinations that maximize the evoked potential and minimize the stimulation artifact 1482. The process may continue to program the best combination of sensing and stimulation configurations in the saved list, based on the filtering, scoring or ranking 1483. The process may continue to provide the evoking stimulation and sensing 1484, and may determine whether the sensed signal is within an input range of the bio-amplifier 1485. If the sensed signal is not within the input range, the current combination of sensing and stimulation configurations may be removed from the list 1486, and the next best combination of sensing and stimulation configurations in the list may be programmed at 1483. If the sensed signal is within the input range of the bio-amplifier, the process may be completed 1487 as the goal of providing a sensed signal within the range of the amplifier has been accomplished.

It is noted that the neurosurgeon may determine the stimulation electrode configuration, and the system may be used to determine an appropriate sensing electrode configuration for that stimulation electrode configuration. The neurosurgeon may determine the sensing electrode configuration, and the system may determine the appropriate stimulation electrode configuration for that sensing electrode configuration. The neurosurgeon may select a target for the neurostimulation and/or a sensing target, and system may determine the appropriate combination of the stimulation electrode configuration and sensing electrode configuration for the selected target(s).

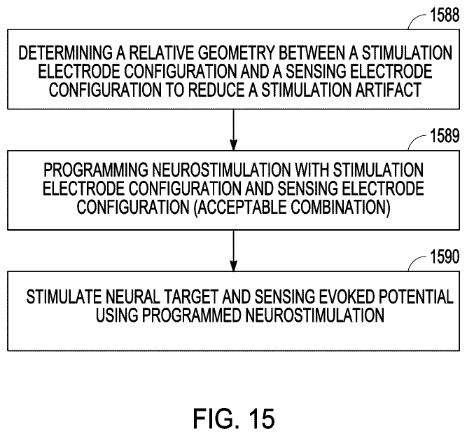

FIG. 15 illustrates, by way of example and not limitation, a method for stimulating a neural target using a determined relative geometry between a stimulation electrode configuration and a sensing electrode configuration to reduce a stimulation artifact. The illustrated method may include determining a relative geometry between a stimulation electrode configuration and a sensing electrode configuration to reduce a stimulation artifact where the stimulation electrode configuration and the sensing electrode configuration are for a neurostimulator having a plurality of electrodes 1588. The determined relative geometry may be used to determine the stimulation electrode configuration and/or the sensing electrode configuration where the stimulation electrode configuration is determined to both stimulate a neural target and cause an evoked potential and the sensing electrode configuration is determined to sense the evoked potential. The neurostimulator may be programmed with the stimulation electrode configuration and the sensing electrode configuration that have the relative geometry to reduce an effect of the stimulation artifact 1589. The neural target may be stimulated 1590 using the neurostimulator programmed with the determined stimulation electrode configuration and the evoked potential may be sensed using the neurostimulator programmed with the determined sensing electrode configuration.

A stimulation artifact is a specific example of an unwanted signal contribution in the sensed signal. A method may include determining stimulation-sensing combination(s) that includes a stimulation electrode configuration for use in delivering stimulation that causes an evoked potential and a sensing electrode configuration for receiving a sensed signal. The sensed signal may include the evoked potential which may be recorded and evaluated. The method may include evaluating the stimulation-sensing combination(s) for reducing unwanted signal contributions in the sensed signal to provide evaluation results, and programming a neurostimulator with the stimulation electrode configuration and the sensing electrode configuration based on the evaluation results. The evaluation of stimulation-sensing combination(s) may include determining a relative physical geometry between a stimulation electrode configuration and a sensing electrode configuration, determining a relative electrical geometry between a stimulation electrode configuration and a sensing electrode configuration, or combinations thereof. The relative electrical geometry may be affected by the active stimulation electrodes, the anodic energy contributions (e.g., anodic fractionalization) of some active electrodes, the cathodic energy contributions (e.g., cathodic fractionalization) of some active electrodes, and the electrodes used for sensing. The combination(s) may be evaluated for improving a signal-to-noise ratio by increasing desired signal and/or decreasing unwanted signal(s) such as a stimulation artifact. Programming the neurostimulator based on the evaluation results may include various stimulation and/or sensing setting along with the stimulation electrode configuration and the sensing electrode configuration.

Each stimulation-sensing combination(s) may be evaluated based on a relative physical geometry and/or a relative electrical geometry between the corresponding stimulation electrode configuration and the corresponding sensing electrode configuration. The evaluation results may include a recommended stimulation-sensing combination with a recommended sensing electrode configuration for a recommended stimulation electrode configuration. The method may further include optimizing at least one of the recommended sensing electrode configuration or the recommended stimulation electrode configuration.

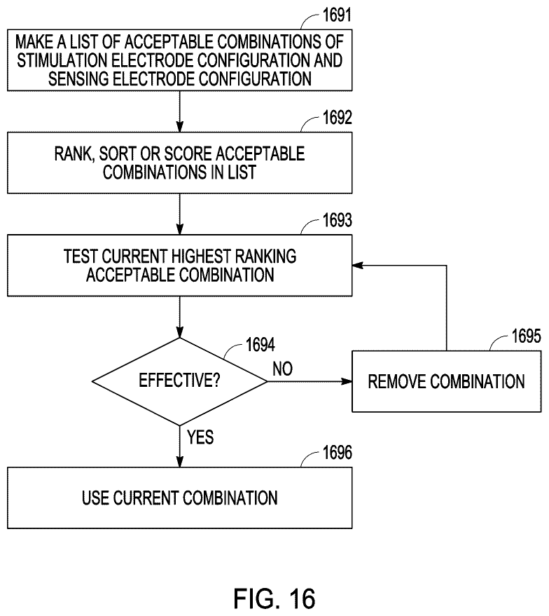

FIG. 16 illustrates, by way of example and not limitation, a method for filtering, scoring or ranking acceptable combinations of stimulation electrode configurations and sensing electrode configurations. At 1691, a list of acceptable combinations of stimulation electrode configuration and sensing electrode configuration may be made. The acceptable combinations in the list may be ranked, sorted or scored 1692. Better combinations provide a better combination for increasing the magnitude of the sensed signal and decreasing the magnitude of the stimulation artifact such that the sensed signal can be better sensed using bio-amplifiers, which may have a limited range. The current best combination may be tested 1693, and it may be determined whether the tested combination is effective 1694. A combination is effective if the sensed signal can be sensed by adequately reducing the amplitude of the stimulation artifact and/or increasing the amplitude of the sensed signal. If the combination is not effective at 1693, the combination is removed from the list 1695 and the next best combination in the list is tested at 1693. If the combination is effective at 1694, the current combination is used 1696.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
determining at least one stimulation-sensing combination that includes a stimulation electrode configuration for use in delivering stimulation that causes an evoked potential and a sensing electrode configuration for receiving a sensed signal, wherein the sensed signal includes the evoked potential;
evaluating the at least one stimulation-sensing combination for reducing unwanted signal contributions in the sensed signal to provide evaluation results; and
programming a neurostimulator with the stimulation electrode configuration and the sensing electrode configuration based on the evaluation results.

2. The method of claim 1, wherein each of the at least one stimulation-sensing combination is evaluated based on at least one of a relative physical geometry or a relative electrical geometry between the corresponding stimulation electrode configuration and the corresponding sensing electrode configuration.

3. The method of claim 1, wherein the evaluation results include a recommended stimulation-sensing combination with a recommended sensing electrode configuration for a recommended stimulation electrode configuration, wherein the method further includes optimizing at least one of the recommended sensing electrode configuration or the recommended stimulation electrode configuration.

4. The method of claim 1, further comprising stimulating a neural target using the neurostimulator programmed with the determined stimulation electrode configuration and sensing the evoked potential using the neurostimulator programmed with the determined sensing electrode configuration.

5. The method of claim 1, wherein:
the neurostimulator is programmed with the determined stimulation electrode configuration and the determined sensing electrode configuration, wherein the programmed neurostimulator is configured to stimulate a neural target using the determined stimulation electrode configuration and sense the evoked potential using the determined sensing electrode configuration;
the neurostimulator includes a lead with a plurality of electrodes organized with longitudinal electrode levels, each longitudinal electrode level includes at least one electrode, and at least one of the longitudinal electrode levels includes segmented electrodes peripherally positioned around the lead in a same longitudinal electrode level;
an electric field is created using electrodes in at least two of the longitudinal electrode positions to generally align an electric field direction along a longitudinal direction of the lead; and
the sensing electrode configuration is configured to use at least two of the segmented electrodes in the same longitudinal electrode level to sense the evoked potential in the sense direction approximately perpendicular to the electric field direction.

6. The method of claim 1, wherein:
the neurostimulator includes at least two leads, each of the at least two leads including a plurality of electrodes organized with longitudinal electrode positions, each longitudinal electrode position includes at least one electrode; and
at least one of the plurality of electrodes in each lead is used to either create an electric field or to sense the evoked potential.

7. The method of claim 1, wherein:
the neurostimulator includes a paddle lead, the paddle lead including a plurality of electrodes; and
at least two of the plurality of electrodes on the paddle lead are configured to create an electric field in an electric field direction and at least two of the plurality of electrodes on the paddle lead are configured to sense the evoked potential in the sense direction approximately perpendicular to the electric field direction.

8. The method of claim 1, wherein the least one the stimulation electrode configuration or the sensing electrode configuration is determined using a biological model, wherein the biological model is configured to model evoked potentials for various sensing directions that are evoked when a neural target is stimulated in various electric field directions.

9. The method of claim 1, wherein the determined at least one stimulation-sensing combination form acceptable combinations of stimulation and sensing electrode configurations, the method further including:
making a list of acceptable stimulation-sensing combinations by determining at least one additional sensing electrode configuration for the stimulation electrode configuration, each of the at least one additional sensing electrode being configured to sense in a corresponding sense direction; and
filtering, scoring or ranking the acceptable combinations in the list based on effectively sensing a sensed evoked potential signal and desirably reducing unwanted signal contributions in the sensed signal.

10. The method of claim 9, wherein the making the list of acceptable stimulation-sensing combinations includes:
determining at least one additional stimulation electrode configuration to create at least one corresponding electric field in at least one corresponding electric field direction for stimulating a neural target; and
determining at least one sensing electrode configuration corresponding to each of the at least one additional stimulation configuration to sense in a corresponding at least one sense direction approximately perpendicular to the direction of the corresponding at least one electric field direction for stimulating the neural target.

11. The method of claim 9, wherein the ranking the acceptable stimulation-sensing combinations includes using a biological model to rank the acceptable stimulation-sensing combinations, wherein the biological model is configured to model evoked potentials for various sensing directions that are evoked when a neural target is stimulated in various electric field directions, and wherein the ranking includes using the biological model to determine at least one of the stimulation electrode configuration or the sensing electrode configuration to desirably increase a magnitude of the sensed evoked potential signal and desirably reduce unwanted signal contributions in the sensed signal.

12. The method of claim 9, wherein:
the neurostimulator includes a lead with a plurality of electrodes organized with longitudinal electrode levels, each longitudinal electrode level includes at least one electrode, and at least one of the longitudinal electrode levels includes segmented electrodes peripherally positioned around the lead in a same longitudinal electrode level; and
the making the list of acceptable stimulation-sensing combinations includes determining different sensing configurations using different combinations of at least two of the segmented electrodes in the same longitudinal electrode level.

13. The method of claim 12, wherein at least a first longitudinal electrode level and a second longitudinal electrode level includes segmented electrodes; and
the making the list of acceptable stimulation-sensing combinations includes determining different sensing configurations using different combinations of at least two of the segmented electrodes in the first longitudinal electrode level and using different combinations of at least two of the segmented electrodes in the second longitudinal electrode level.

14. The method of claim 9, further comprising:
programming a highest ranked acceptable stimulation-sensing combination in the list into the neurostimulator as a current configuration;
stimulating a neural target and sensing the evoked potential using the programmed neurostimulator; and
determining whether the sensed evoked potential is within an input range of a bio-amplifier used to amplify the sensed evoked potential.

15. The method of claim 14, wherein the sensed evoked potential is determined to not be within the input range of the bio-amplifier, the method further including removing the current configuration from the list and programming a remaining highest ranked acceptable stimulation-sensing combination in the list into the neurostimulator as a subsequent current configuration.

16. The method of claim 1, wherein the programmed stimulation electrode configuration is configured to provide an electric field with an electric field direction and the programmed sensing electrode configuration is configured to sense the evoked potential to favor a sense direction that is approximately perpendicular to the electric field direction.

17. A non-transitory machine-readable medium including instructions, which when executed by a machine, cause the machine to perform a method comprising:
determining at least one stimulation-sensing combination that includes a stimulation electrode configuration for use in delivering stimulation that causes an evoked potential and a sensing electrode configuration for receiving a sensed signal, wherein the sensed signal includes the evoked potential;
evaluating the at least one stimulation-sensing combination for reducing unwanted signal contributions in the sensed signal to provide evaluation results; and
programming a neurostimulator with the stimulation electrode configuration and the sensing electrode configuration based on the evaluation results.

18. The non-transitory machine-readable of claim 17, wherein the method further includes:
the neurostimulator includes at least two leads, each of the at least two leads including a plurality of electrodes organized with longitudinal electrode positions, each longitudinal electrode position includes at least one electrode; and
at least one of the plurality of electrodes in each lead is used to either create an electric field or to sense the evoked potential.

19. The non-transitory machine-readable of claim 17, wherein the least one the stimulation electrode configuration or the sensing electrode configuration is determined using a biological model, wherein the biological model is configured to model evoked potentials for various sensing directions that are evoked when a neural target is stimulated in various electric field directions.

20. A system, comprising a programmer for a neurostimulator wherein the neurostimulator has a plurality of electrodes, the programmer including a processor and a memory including instructions which when executed by the processor perform a method that includes:
determining at least one stimulation-sensing combination that includes a stimulation electrode configuration for at least some of the plurality of electrodes for use in delivering stimulation that causes an evoked potential and a sensing electrode configuration for at least some of the plurality of electrodes for receiving a sensed signal, wherein the sensed signal includes the evoked potential;
evaluating the at least one stimulation-sensing combination for reducing unwanted signal contributions in the sensed signal to provide evaluation results; and
programming the neurostimulator with the stimulation electrode configuration and the sensing electrode configuration based on the evaluation results.
